(19) United States
(12) Patent Application Publication
(10) Pub. No.: US 2024/0180416 A1
(43) Pub. Date: Jun. 6, 2024

(54) SYSTEM AND METHOD FOR PROVIDING VISUAL FIELD TESTS

(71) Applicant: OLLEYES, INC., Summit, NJ (US)

(72) Inventors: Alberto O. GONZALEZ GARCIA, Randolph, NJ (US); Freddy Salomon MORGENSTERN, Wayne, PA (US)

(73) Assignee: OLLEYES, INC., Summit, NJ (US)

(21) Appl. No.: 18/553,761

(22) PCT Filed: Apr. 21, 2022
(86) PCT No.: PCT/US22/25677

§ 371 Date: Oct. 3, 2023

Related U.S. Application Data
(60) Provisional application No. 63/177,605, filed on Apr. 21, 2021.

(51) Int. Cl. A61B 3/024; A61B 3/00; A61B 3/113

(52) U.S. Cl. CPC A61B 3/024; A61B 3/0091; A61B 3/113; A61B 2503/06

(57) ABSTRACT

Embodiments of the invention are directed towards systems, methods and computer program products for providing improved eye tests. Such tests improve upon current eye tests, such as visual field tests, by incorporating a gamification of the testing process, software mediated guidance to the patient or practitioner such that more accurate results of the eye tests are obtained. Furthermore, through the use of one or more trained machine learning or predictive analytic systems, multiple signals obtained from sensors of a testing apparatus are evaluated to ensure that the eye test results are less error-prone and provide a more consistent evaluation of a user's vision status. As it will be appreciated, such error reduction and user guidance systems represent technological improvements in eye tests and utilize non-routine and non-conventional approaches to the improvement and reliability of eye tests.

### CROSS REFERENCE TO RELATED APPLICATION

The present application claims priority to U.S. Patent Application Ser. No. 63/177,605 filed Apr. 21, 2021, which is hereby incorporated by reference in its entirety.

### FIELD OF THE INVENTION

The invention generally relates to systems and methods for the testing the eye of a subject. More particularly, the invention relates to a system and method for performing interactive visual field tests on a subject.

### BACKGROUND OF THE INVENTION

Eye testing, such as visual field testing, are common approaches to measuring a subject's vision. For instance, visual field testing (VF) is concerned with measuring the clearness of a subject's vision outside the central fixation. Visual Field tests are usually carried out by a health care professional. While most VF tests are performed on adult patients, it is necessary to perform VF tests on adolescent and children. Typically, the VF tests used on children are the same ones used in the adult population. Doing so fails to take into account the differences between adult and child physiology, mental states and conceptual understandings of the rationale behind obtaining accurate measurements. Thus, what is needed in the art is a more robust VF test, such as one that incudes appropriate guidance systems. Such guidance systems aid in conducting eye test assessments in pediatric population. Furthermore, what is needed in the art are systems, platforms and apparatus that incorporates interactive and dynamic functionality to ensure the attentiveness of patients, such as pediatric patients, during the presentation of stimulus as part of a visual test.

### SUMMARY OF THE INVENTION

Embodiments of the invention are directed towards systems, methods and computer program products for providing improved eye tests. Such tests improve upon current eye tests, such as visual acuity tests, by incorporating virtual reality, test gamification and software mediated guidance to the patient or practitioner such that more accurate results of the eye tests are obtained. Furthermore, through the use of one or more trained machine learning or predictive analytic systems, multiple signals obtained from sensors of a testing apparatus are evaluated to ensure that the eye test results are less error-prone and provide a more consistent evaluation of a user's vision status. As it will be appreciated, such error reduction and user guidance systems represent technological improvements in eye tests and utilize non-routine and non-conventional approaches to the improvement and reliability of eye tests.

In one particular implementation the present apparatus, systems and computer implemented methods described herein are utilized to provide an improved visual field test, the improved visual field test comprising, at least one data processor comprising a virtual reality engine and at least one memory storing instruction which is executed by the at least one data processor, the at least one data processor configured to present a virtual assistant in virtual reality, wherein the virtual assistant presents to a patient a set of instructions for the visual field test. In a further implementation, the system described is configured to receive from the patient at least one response when the patient views at least one stimulus, wherein the response comprises a selection of a position of the at least one location of a stimulus. Using this information, the system described repeats these steps at least y times, where y is an integer greater than 2, until the patient indicates that he/she cannot identify any stimulus. Based on the patient's response, if a percentage of responses labeled as correct is less than the percentage expected to be correct based on a historical value for the patient's visual field score or an estimated percentage of correct choices based on a probability score, the explanation is modified to provide a second set of instructions selected from a library instructions. Based on the patient's response, if a percentage of responses labeled as correct is greater than or equal to the percentage expected to be correct based on a historical value for the patient's visual field score or an estimated percentage of correct choices based on a probability score, a visual field score is calculate.

### BRIEF DESCRIPTION OF THE DRAWINGS

Embodiments of the invention are illustrated in the figures of the accompanying drawings which are meant to be exemplary and not limiting, in which like references are intended to refer to like or corresponding parts, and in which:

FIG. 1 illustrates a block diagram of a system for implementing visual field and other eye tests according to one embodiment of the present invention;

FIG. 2 illustrates a generated virtual environment according to one embodiment of the present invention; and

FIG. 3 illustrates a function diagram of various modules according to one embodiment of the present invention.

### DETAILED DESCRIPTION OF CERTAIN EMBODIMENTS OF THE INVENTION

By way of overview, various embodiments of the systems, methods and computer program products described herein are directed towards devices to conduct improved eye tests. For example, the systems, methods and computer program products described herein can be utilized to investigate afferent function in children using perimetry. In a particular implementation, the improved eye tests described herein are directed to devices for providing improved visual field tests. Furthermore, in one or more particular implementations, the visual field testing devices described herein are configured to enable a user (such as a subject or patient) to self-administer the visual field test. In this manner, the user is freed from the time and costs associated with scheduling and physically visiting a physician or medical facility to obtain a visual field test. Likewise, the provided systems provide improved attention to task in the administered. In some embodiments this is accomplished by providing provide one or more interactive and engaging game scenario to a user, which are likely to increase attention to task in the subject and thus result in a more accurate test. Such improved attention provides for less variability between and among subjects.

In one or more embodiments, the patient is a pediatric patient. In some embodiments, the pediatric patient is aged 21 years or younger. In some embodiments, the pediatric patient is an adolescent between 10 and 21 years old. In some embodiments, the pediatric patient is a child less than 10 years old. In some embodiments, the pediatric patient is greater than or equal to 5 years old. In some embodiments, the patient is an adult that is greater than 21 years old.

In one or more implementations of the systems, apparatus and computer program products described herein, the improved platform for administering visual field tests includes providing an altered field of vision device to a user that that incorporates one or more virtual interactive environments, such as games, that provide iterative guidance to the user. The devices and associated processes coordinate to provide visual field tests that are accurate and reliable.

In one or more further implementations, the nature, type and content of the dynamic environment provided to the user is based on the output of one or more machine learning systems. Additionally, through the use of one or more trained machine learning based systems, the results of the pediatric eye test can be interpreted so as to provide more accurate measurements of the patient's current visual state by avoiding or reducing measurement errors.

In one or more further implementations, the interactive visual field test includes one or more processors configured to receive and/or implement data obtained from one or more machine learning systems. For instance, one or more machine learning modules are configured to generate predictive data that determines the placement or movement of one or more dynamic elements within a displayed visual field test. Such predictive data is based on, among other data factors, a training set or corpus of data that evaluates the placement of visual elements and the corresponding patient score of a visual test. Furthermore, such machine learning systems improve the overall experience of a user such that the visual field test is more streamlined, informative, less stressful and able to produce more consistent results. Such systems and approaches described herein represent improvements in the technological art of visual field testing through the use of non-routine and non-conventional approaches that improve the functionating of visual field testing platforms.

Various objects, features, aspects and advantages of the subject matter described herein will become more apparent from the following detailed description of particular implementations.

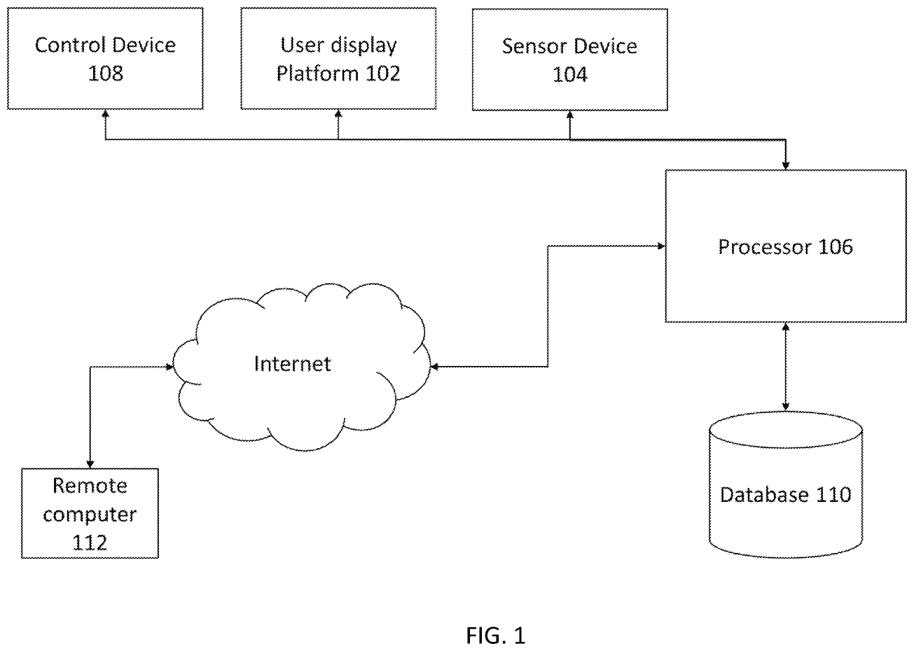

Turning now to FIG. 1, a schematic of an eye testing platform system is provided. The foregoing elements provided in FIG. 1 are components or devices that are linked, connected, networked or otherwise interfaced with one another (shown in solid lines) such that data generated or residing on one or more of the components can be passed or exchanged to a different component. For example, bi-directional data transfers are indicated by the solid lines connecting each of the labeled components. It will be appreciated that the communication linkages between the components provided can be wired or wireless and that each component includes the necessary hardware and software to enable wired or wireless communication between the provided elements or components.

In one or more configurations of the pediatric eye testing system and methods described herein, a user display platform, or display unit, 102 is provided to a subject. In a particular implementation, the subject is an adolescent. The user display platform 102 is configured to provide a form factor, size or other dimensions suitable for use by a child or adolescent. For example, in one implementation, the user display platform 102 is the Olleyes VisuALL (OV) or another make and model of an automated static threshold perimeter.

In one or more implementations, the user display platform 102 is configured to receive user input data from a user in response to carrying out a visual field test. For example, in some embodiments, and in no way limiting the scope of this disclosure, the user display platform 102 is a virtual reality (VR) or augmented reality (AR) device the provides a mediated field of view to a user. For example, a user may wear a pair of goggles, a pair of glasses, a monocle or other display device that allows for a display to be presented to a user where such a display provides a semi- or completely virtual visual environment to the user. In one or more implementations, the user display platform 102 includes a screen, monitor, display, LED, LCD or OLED panel, augmented or virtual reality interface or an electronic ink-based display device that provides the visual display to the wearer. By way of further example, the display element (such as stereoscopic LED or LCD panels) incorporated into a user display platform 102 is configured to generate and provide one or more icons, graphics, graphical user interfaces or computer-generated imagery to the wearer or user.

In one or more implementations, the pediatric eye testing system further includes one or more sensors or state sensing devices 104. For example, one or more sensors 104 are integrated into the form factor of the user display platform 102 and are configured to obtain data measurements of the user or wearer during various operations. In one particular implementation, the sensors 104 are configured to determine or generate data that corresponds to the position and movement of a user or wearer of the user display platform 102. For example, the user display platform 102 includes one or more sensors 104 configured as orientation tracking units, structured light scanners, IR position trackers, magnetometers, pressure sensors, gyroscopes, accelerometers, as well as the necessary power supplies and data storage memories and associated components to implement such displays and sensors. In one or more implementations, the sensors 104 devices are configured to output data to one or more local or remote data processing devices, processors or computers, such as processor 106. In particular configurations, the sensors are implemented to track the eye movement of the wearer during an eye test. In such configurations, the sensors 104 are directed towards the face of the wearer while the wearer is wearing the user display platform 102.

In one or more further implementations, the pediatric eye testing system further includes one or more control devices 108. In some embodiments, the control devices 108 are configured to send data to one or more processors, such as but not limited to, processor 106. In one implementation, the control devices 108 are one or more keyboards, computer mouses, joysticks, game pads, Bluetooth based input devices or other devices configured to receive user input or commands. For example, a data processor 106 is configured to receive data from one or more control devices 108. In response, the processor (such as processor 106) causes one or more graphical elements presented on the user display platform 102 to be adjusted, moved, created or removed. Alternatively, the processor 106 is configured to cause a graphical element that is being displayed by the user display platform 102 to simulate movement or repositioning of that element in response to user input.

The data gathered by the processor 106 can be packaged and uploaded to a persistent data store, which may be local or remote to the control device, e.g., to serve as supporting data with regard to the safety or efficacy of a particular visual field test.

In one or more implementations, the pediatric eye testing system further includes one or more processors or computer elements 106. For example, a processor when used generally throughout, and not exclusively when referring to the pediatric eye testing system further, can be a computer or discrete computing element such as microprocessor. In one or more particular implementations, the processor 106 is incorporated into one a desktop or workstation class computer that executes a commercially available operating system, e.g., MICROSOFT WINDOWS, APPLE OSX, UNIX or Linux based operating system implementations. In another implementation, the processors or computers 106 are located or configured as a cloud or remote computing cluster made of multiple discrete computing elements, such as servers. Such a cloud computing cluster is available on an as needed basis and can provide a pre-determined level of computing power and resources. In accordance with alternative embodiments, the processors or computer of the pediatric eye testing system can be a portable computing device such as a smartphone, wearable or tablet class device. For example, in some embodiments, the processor 106 of the pediatric eye testing system is an APPLE IPAD/IPHONE mobile device, ANDROID mobile device or other commercially available mobile electronic device configured to carry out the processes described herein. In other embodiments, the processor 106 of the pediatric eye testing system comprises custom or non-standard hardware configurations. For instance, the processor may comprise one or more micro-computer(s) operating alone or in concert within a collection of such devices, network adaptors and interfaces(s) operating in a distributed, but cooperative, manner, or array of other micro-computing elements, computer-on-chip(s), prototyping devices, “hobby” computing elements, home entertainment consoles and/or other hardware.

The processor 106, as well as the user display platform 102, sensor device 104 and control device 108 can be equipped with or be in communication with a persistent memory (not shown) that is operative to store the operating system or the relevant computer or processor in addition to one or more additional software modules, such as those described herein that relate to implementing visual tests and providing for the described functionality in accordance with embodiments described herein.

In one or more implementations, the persistent memory includes read only memory (ROM) and/or a random-access memory (e.g., a RAM). Such computer memories may also comprise secondary computer memory, such as magnetic or optical disk drives or flash memory, that provide long term storage of data in a manner similar to the persistent storage. In accordance with one or more embodiments, the memory comprises one or more volatile and non-volatile memories, such as Programmable Read Only-Memory (“PROM”), Erasable Programmable Read-Only Memory (“EPROM”), Electrically Erasable Programmable Read-Only Memory (“EEPROM”), Phase Change Memory (“PCM”), Single In-line Memory (“SIMM”), Dual In-line Memory (“DIMM”) or other memory types. Such memories can be fixed or removable, as is known to those of ordinary skill in the art, such as through the use of removable media cards or similar hardware modules. In one or more embodiments, the memory of the processor 106 provides for storage of application program and data files when needed by a processor or computer. One or more read-only memories provide program code that the processor or computer 106 of the eye testing system reads and implements at startup or initialization, which may instruct a processor associated therewith to execute specific program code from the persistent storage device to load into RAM at startup.

In one embodiment provided herein, the modules stored in memory utilized by the eye testing system includes one or more software program code and data that are executed or otherwise used by one or more processors integral or associated with the eye testing system thereby causing a processor thereof to perform various actions dictated by the software code of the various modules. For instance, the eye testing system is configured with one or more processors that are configured to execute code. Here, the code includes a set of instructions for evaluating and providing data to and from the user display platform 102, control devices 108 and sensor devices 104.

Building on the prior example, the pediatric eye testing system at startup retrieves initial instructions from ROM as to initialization of one or more processors. Upon initialization, program code that the processor retrieves and executes from ROM instructs the processor to retrieve and begin execution of a visual test or calibration process code. The processor, such as processor 106, begins execution of the visual or eye test application program code, loading appropriate program code to run into RAM and presents a user interface to the user that provides access to one or more functions that the program code offers. According to one embodiment, the visual test or eye test application program code presents a main menu after initialization that allows for the creation or modification of the user's desired test, customization parameters, information, testing plans, prior test results, and other information or protocols that are relevant to a user. While reference is made to code executing in the processor, it should be understood that the code can be executed or interpreted or comprise scripts that are used by the processor to implement prescribed routines.

In accordance with certain embodiments, one or more processors of the eye testing system is also in communication with a persistent data store, or database, 110, the one or more processors being located remote from the remote persistent data store 110 such that a processor 106 is able to access the remote persistent data store 110 over a computer network, e.g., the Internet, via a network interface, which implements communication frameworks and protocols that are well known to those of skill in the art.

In one configuration, the remote persistent data store 110 is connected to the processor 106 via a server or network interface and provides additional storage or access to user data, community data, or general-purpose files or information. The physical structure of the remote persistent data store 110 may be embodied as solid-state memory (e.g., ROM), hard disk drive systems, RAID, disk arrays, storage area networks (“SAN”), network attached storage (“NAS”) and/or any other suitable system for storing computer data. In addition, the remote persistent data store 110 may comprise caches, including database caches and/or web caches. Programmatically, the remote persistent data store 110 may comprise flat-file data store, a relational database, an object-oriented database, a hybrid relational-object database, a key-value data store such as HADOOP or MONGODB, in addition to other systems for the structure and retrieval of data that are well known to those of skill in the art.

In addition to a remote persistent data store 110, the processor 106 may connect to one or more remote computing devices 112 over a network connection. Such computing devices are configured to exchange data with the processor 106.

In one or more implementations, the remote persistent data store 110 can provide or provide access to one or more machine learning models. For example, the persistent data store 110 is configured to store a pre-trained neural network or other machine learning or artificial intelligent model or agent. Here, such as model is configured to accept data provided by the processor 106, user display 102, sensor 104 or control device 108, of any combination thereof, and provide output data in response thereto. In another implementation, the remote computer 112 is configured to host such a machine learning system, model or platform and provide access thereto to the processor 106. For example, the remote computer 112 is configured to implement such artificial intelligence systems as a separate predictive system that is accessible to the eye test system. In one particular implementation, the processor 106 is configured to access a stored machine learning model from the remote computer 112 or database 110 and provide input data or configuration data to the accessed model. In turn, the data and predictive models stored in the remote data store 110 or remote computer 112 are used to generate new data or instructions to be executed by the processor 106 or the user display platform 102 that causes changes to the display presented to a user in connection with the tasks being performed. In an alternative implementation, the processor 106 is configured to access and query a local (such as operating within the memory of the processor) instance of a neural network or other machine learning model.

In one or more particular implementations of the systems and methods described herein, the remote computer 112 is configured as a predictive system. In another configuration, the processor 106 is configured to implement a predictive system that updates or controls aspects of the eye test. In one or more configurations the predictive system includes two (2) neural networks. In one or more alternative configurations, additional neural networks can be used. It will be appreciated that neural networks can include both untrained, self-learning or directed, or pre-trained neural networks. For example, each neural network accessed or utilized by the remote computer 112, processor 106 or stored in the remote datastore 110 can be trained using training databases.

By way of a further example, in one particular implementation, the user display platform 102, control device 108 and sensor device 104 can be used to train a neural network. Here, a population of users are provided with a number of visual prompts, commands and instructions corresponding to one or more different eye tests. Each user's performance (and data generated from the control device 108 and sensor device 104 during such performance) are correlated to the user's score on the various eye tests. By way of a further example, such correlations can associate the control device 108 and sensor device 104 data measurements, as well as the input provided by the user display platform 102 with a predictive outcome on one or more visual tests. In one particular implementation, the training data can asynchronously or synchronously process the incoming features and labels and store it in the training database for offline training.

In a further example, the predictive system can use inputs obtained during the eye examination or test, such as but not limited to data received from the control device 108 and sensor device 104. By way of non-limiting example, the following inputs can include data or values corresponding to a given step or portion of an eye test; the vector of movement of a control device 108 or other hardware device; the current duration of the test; one or more values corresponding to the speed of movement of the subject's eyes, head or control device 108; one or more values corresponding to fixation or placement of a cursor (generated on the display provided to the user through the user display platform 102); one or more values corresponding to a given amount of time without the system detecting a response from the subject; one or more values corresponding to a given amount of time without the system detecting a control device 108 (such as a handpiece) movement; one or more values corresponding to a given position of the headset; one or more values corresponding to a detection of a user or subject eye state (examples of the subject eye state can include, but are not limited to, eyes open, eyes closed, or ptosis) by the eye-tracking system (ETS) during a given amount of time; one or more values corresponding to an excessive amount of fixation loses (for example, when the eyes are not focused on a point on the screen and are looking someplace else); one or more values corresponding to when a non-expected number of incorrect responses are detected; one or more values corresponding to an excessive amount of incorrect stimulus detection events; one or more values corresponding to an incorrect selection of stimulus; one or more values corresponding to an incorrect eye position; one or more values corresponding to a given position of the user display platform 102.

In each of the foregoing examples, the term “incorrect” or “excessive” can be established by way of a pre-set threshold value for the given data feature. For example, the value for incorrect direction or vector of movement can be established through one or more statistical analysis on the anticipated amount of incorrect direction. Similar statical values or pre-determined thresholds are known, understood and appreciated by those possessing an ordinary level of skill in the requisite art.

In one or more implementations, the predictive system described herein is configured to take one or more actions in response to the output of the decision tree. Thus, in a particular implementation, the predictive system configures the stimulus presentation (such as, but not limited to the elements provided to the user as part of the user display platform 102) to revise or alter information dynamically in response to the user's own actions. Alternatively, the responses can be dynamically generated based, in part, on the current state values.

By way of non-limiting example, the predictive system can undertake a configuration of the pediatric eye test system in order to provide a personalized or dynamically generated test flow based on said quality criteria. For example, the pediatric eye test system can be configured by one or software modules to avoid providing a user with more than a threshold amount of prompts in response to an incorrect stimuli selection.

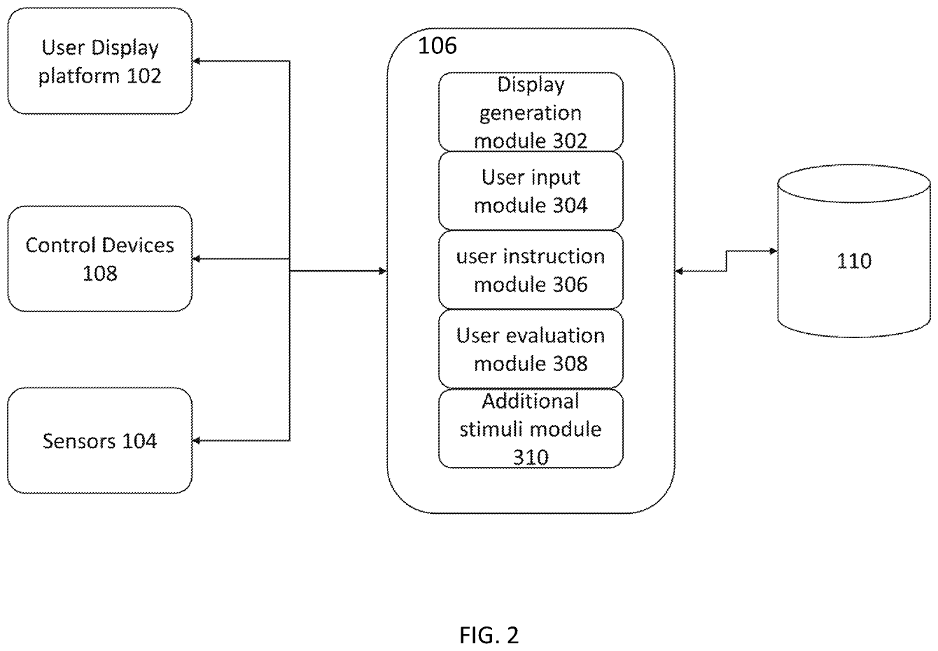

In one or more further implementations, a pediatric eye test system is provided that is configured by one or more modules executing as code in a processor (such as processor 106) to provide an interactive and engaging game scenario to a user. As noted, game scenarios are likely to increase attention to task in the subject and thus result in a more accurate test. For example, as shown in FIG. 2, the processor 106 is configured by a display generation module 302. Here, the display generation module 302 is configured as code that instructs the processor 106 to send a game scenario to the virtual, augmented or mixed reality hardware (such as, but not limited to user display platform 102).

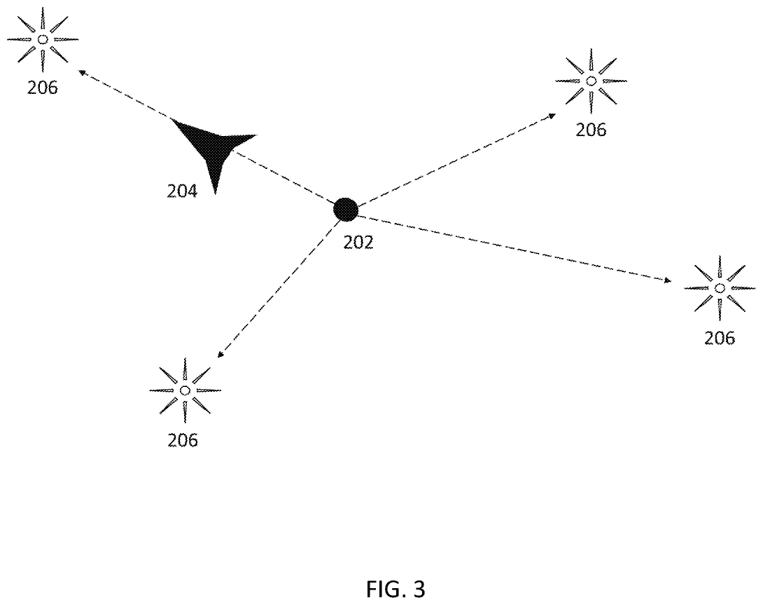

Here, processor 106 is configured to cause a game scenario to be presented to the subject so as to increase subject engagement with the testing task. For example, as shown in FIG. 3, a pediatric visual field test is administered to an adolescent. The user display platform 102 is worn or otherwise presented to the user. Here, the processor 106 is configured by the display generation module 302 to cause the user display platform 102 to present at least three visual elements for dynamic interaction by the user: a fixation target 202, a cursor 204 and one or more stimuli 206. In some embodiments the one or more stimuli 206 are a part of a stimulation matrix. In the foregoing implementation, the visual elements are configured as a space travel simulator. In one or more further implementations, the processor 106 configures the fixation target 202 to be a planet. As show, in FIG. 2, a fixation point (for example, Mars, a red planet) is presented on the screen of the user display platform 102 in front of a dark background (shown as a star field). While the foregoing example provides a space travelling scenario to the user, it should be understood that the fixation target could also be automobile, an animal or any other cartoonish representation of a real or fictitious object or animal. Likewise, the background can be any color or representation, as long as there is sufficient contrast so as to be able to distinguish the fixation point 202 from the background. Similarly, there the background should be a color that allowed for sufficient contrast between it and the stimuli 206.

Here, the processor 106 is configured by a user input module 304 that updates the position of the displayed cursor 204 in response to input received from the one or more control devices 108. By way of example, the user input module 304 configures processor 106 to allow the movement of the cursor 204 to be controlled by a handpiece (control device 108) that is wirelessly connected to the user display platform 102. As noted, in the exemplary embodiment, the displayed setting is an outer space setting, with a planet as a fixation point, and the cursor represented by a spacecraft. This game scenario should not be restricted to a such a scenario, and it will be appreciated that any other game scenario where the cursor can be depicted as a representation of a movable object. For example, the cursor 204 is represented by an object that is understood and appreciated to be mobile or movable. For instance, the cursor could be represented as an animal, automobile or other object that is mobile.

In further implementation, the processor 106 is configured to receive user input from the user using the control device 108 to control the movement of the cursor 204. In the illustrated implementation, the cursor 204 is configured as a spacecraft, but can take any form suitable for the scenario being displayed. Here, the processor 106 is configured by a user instruction module 306 to provide instructions to the user to inform the user that the cursor 204 (for example the spacecraft) can be moved around the dark background relative to the fixation point 202. For instance, where the control device 108 is Bluetooth connected handpiece (such as a joystick), an audio or visual instruction can be provided or displayed to the user. Here, the displayed or provided instructions are designed to inform the user that the control device 108 can be used to move the cursor 204 to the fixation point 202. For example, the system described herein encourages the user to move and land the spacecraft (cursor 204) over Mars (fixation point 202) by showing a path or vector (in dashed lines).

Next, the processor 106 receives data input from the control device 108 as well as the sensor 104 while the user is attempting to move (as shown in the dashed line) the cursor 204 to the fixation point 202. For example, one or more eye tracking sensors is used to track the eye movement, pupil dilation and/or other biometric parameters of the user while moving the cursor 204 to the fixation point 202. For example, one or more eye tracking sensors are configured to determine the location on a display of the user display platform 102 where the user is focusing. In this arrangement, users with limited manual dexterity are able to correctly identify the fixation point 202 without the use of the control device 108.

In one arrangement, the processor 106 is configured by a user evaluation module 308 to determine that the cursor 204 has been placed on the fixation point 202. In one arrangement, the processor 106 is configured by the user evaluation module 308 to compare the pixel values of the displayed graphical elements representing the cursor 204 and the fixation point 202. Where the pixel coordinates of the two elements substantially overlap, the processor 106 is configured by code executing therein to determine that the user has successfully identified the fixation point 202. Alternative approaches to determining that the user has identified the fixation point are also understood.

Once the processor 106 has determined that the user has accurately identified the fixation point, the user is then presented with one or more additional stimuli 206. For example, the processor 106 is configured by the additional stimuli module 310 to cause the user display platform 102 to present several light stimuli 206 within a given radius (e.g., 10, 24 or 30 degrees) of the fixation point. In the provided example, the stimulus 206 are depicted as “shining stars”. In one particular implementation, the processor 106 is configured to instruct the user to quickly move the spacecraft (cursor 204) toward the shining star (stimuli 206). In one embodiment, the processor selects a first shining star and causes the display of the shining star to display a particular color, hue, size, or implement an animation that cycles through multiple colors, sizes, or hues (or a combination thereof).

The processor 106 is configured to record whether a successful identification event occurs. For instance, the user evaluation module 308 configured the processor to determine if the subject moves the spacecraft toward the right position of the additional stimuli 206. In one arrangement, the intended destination of the spaceship (cursor 204) will blink or change color. This visual indication clarifies for the user the correct stimuli to focus upon. In this manner, the processor 106 is configured to evaluate whether the response is a false response or not. By way of example, the movement of the cursor 204 to the correct stimuli is unlikely to be a coincidence. Therefore, information about the eye state or other parameters of the user can be determined based on the correct or incorrect selection of the indicated stimulus 206. However, it is possible for there to be coincidences in the selection of stimulus. As such, to provide a more robust confidence level that the user has affirmatively selected the indicated stimulus, the eye tracker sensor (or one or more other sensors 104) is configured to confirm that the user is looking at the same stimulus that has been selected by the cursor 204. In this manner, the processor 106 is configured to confirm that the movement of the cursor 204 to the correct stimuli 206 was not a coincidence. The processor 106 is configured by the additional stimuli module 310 to iteratively provide additional stimuli to the user and evaluate the user's ability to intersect the cursor 204 with the additional stimuli. Such a process is repeated until all the predefined locations of stimuli 206 have been presented to the user and the threshold values (based on one or more sensor measurements of the eyes of the subject when looking at the relevant stimulus) have been calculated at each of those locations.

In some embodiments, the system, method and apparatus is configured to provide a pupil segmentation image analysis and/or an anterior segment image analysis. Pupil segmentation is critical for line-of-sight estimation based on the pupil center method. Anterior segment imaging allows for an objective method of visualizing the anterior segment angle. Two of the most commonly used devices for anterior segment imaging include the anterior segment optical coherence tomography (AS-OCT) and the ultrasound biomicroscopy (UBM).

Additional further implementations of the systems methods and apparatus described herein are understood. For example, and in no way limiting, the following specific, non-limiting numbered implementations are particular configurations of the subject matter described herein.

Implementation 1. A system for a visual field test comprising, at least one data processor comprising a virtual reality, an augmented reality or a mixed reality engine and at least one memory storing instruction which is executed by at least one data processor, at least one data processor configured to:
(i) present a game in virtual reality, augmented reality or mixed reality, wherein the game is presented to a patient as a method of testing the visual field of a given patient,
(ii) provide a fixation point and a set of n stimuli wherein n in some embodiments is 1 but can be greater than or equal to 2 in other embodiments, each fixation having a (a) specified size, (b) shape and (c) luminance, wherein the luminance of the fixation point is greater than the luminance of a background,
(iii) receive from the patient at least one response when the patient views at least one stimulus, wherein the response comprises moving the cursor toward the position of the viewed stimulus;
(iv) repeat steps (ii) to (iii) at least y times, where y is greater than 2, until the patient indicates that he/she cannot identify any stimulus having a size smaller or luminance dimmer than the last stimulus the patient responded to in step (iii);
(v) repeat steps (i) to (iv), wherein the explanation in step (i) is modified, based on the patient's response, to provide a second set of instructions selected from a library of instructions if a percentage of responses in step (iii) labeled as correct is less than the percentage expected to be correct based on a historical value for the patient's visual field score or an estimated percentage of correct choices based on a probability score; or
(vi) calculate a visual field score if a percentage of responses in step (iii) labeled as correct is greater than or equal to the percentage expected to be correct based on a historical value for the patient's visual field score or an estimated percentage of correct choices based on a probability score.

While this specification contains many specific embodiment details, these should not be construed as limitations on the scope of any embodiment or of what can be claimed, but rather as descriptions of features that can be specific to particular embodiments of particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this specification have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results.

Publications and references to known registered marks representing various systems are cited throughout this application, the disclosures of which are incorporated herein by reference. Citation of any above publications or documents is not intended as an admission that any of the foregoing is pertinent prior art, nor does it constitute any admission as to the contents or date of these publications or documents. All references cited herein are incorporated by reference to the same extent as if each individual publication and references were specifically and individually indicated to be incorporated by reference.

While the invention has been particularly shown and described with reference to a preferred embodiment thereof, it will be understood by those skilled in the art that various changes in form and details may be made therein without departing from the spirit and scope of the invention. As such, the invention is not defined by the discussion that appears above, but rather is defined by the claims that follow, the respective features recited in those points, and by equivalents of such features.

What is claimed is:

1. A system to administer a Visual Field test to a pediatric patient, the system comprising:
a display unit configured to display visual information to the pediatric patient, wherein the display unit is secured to a portion of the head of the pediatric patient and depicts one or more visual elements;
a control device, configured to control the one or more visual elements depicted on the display unit; and
one or more sensors configured to track movement of the pediatric patient during administration of the visual field test.

2. The system of claim 1,
further wherein:
the display unit is a virtual, augmented or mixed reality goggles configured to provide the visual field test; and
the one or more sensors are configured to monitor the pediatric patient during administration of the visual test, wherein the one or more sensors is disposed within the goggles and configured to track one or more eye-movements of the pediatric patient;
and further comprising:
at least one data processor; and
at least one memory storage device.

3. The system of claim 2, wherein the processor is configured to:
(i) show a fixation target that simulates a stationary object;
(ii) provide a mobile cursor that simulates a mobile object capable of independent motion and that is controllable in response to input received from the pediatric patient by a control device;
(iii) receive from the pediatric patient at least one first response, wherein the at least one first response comprises moving the mobile object, using the control device, in the direction of the stationary object;
(iv) provide instructions to move the mobile object to be superimposed over the stationary object;
(v) provide a set of n stimuli, wherein n is greater than or equal to 2 wherein each stimulus in the set of n stimuli has a specified (a) size, (b) shape, (c) contrast and (d) luminance, wherein the luminance of each stimulus is greater than the luminance of a background;
(vi) receive from the pediatric patient at least one second response when the pediatric patient views at least one stimulus, wherein the at least one second response comprises moving the mobile object, using the control device, in the direction of the one stimulus;
(vii) repeat steps (ii) to (vi) at least y times, where y is greater than 2, until the patient indicates that a lowest stimulus intensity has been seen; and
(viii) calculate a visual field score if a percentage of responses in step (vii) labeled as correct is greater than or equal to the percentage expected to be correct based on a historical value for the patient's retinal sensitivity score or an estimated percentage of correct choices based on a probability score.

4. The system of claim 3, wherein the control device is a wireless connected handpiece and the mobile object is controlled by the wireless connected handpiece and is used by the system to receive the at least one response from the pediatric patient.

5. The system of claim 3, wherein the at least one first response of moving the mobile object is used by the system to understand whether the subject is fixating in the stationary object or not.

6. The system of claim 3, wherein said stimuli are presented in several locations throughout the pediatric patient's visual field.

7. The system of claim 3, wherein said movement of the mobile object toward the stimuli is recorded and is a vector movement and is used by the system to confirm whether the subject did or did not see the stimulus.

8. The system of claim 7, wherein knowing the vector of the mobile object movement detects potential false positives or negatives.

9. The system of claim 2, wherein said eye tracking system is used to control a gaze fixation.

10. The system of claim 3, wherein one of the said sensors is an eye tracking system configured to:
(i) turn ON the eye tracking sensors at a time in the range of from 1 to 5 milliseconds before providing the set of n stimuli, wherein the eye tracking sensors produce a fixation status;
(ii) analyze the fixation status before providing the set of n stimuli;
(iii) detect eye fixation on the fixation target;
(iv) continue the Visual Field test without modifications, if the eye tracking system detects eye fixation on the fixation target; and
(v) adjust the position of the set of n stimuli a before presenting the set of n stimuli if the eye tracking system detects eye fixation on a position other than fixation target, to match the said new detected position of the eye.
